(19) United States
(12) Patent Application Publication
(10) Pub. No.: US 2009/0143652 A1
(43) Pub. Date: Jun. 4, 2009

(54) Apparatus and Method for Measuring, Recording and Transmitting Primary Health Indicators

(75) Inventors: Ian Warburton, Poquott, NY (US); Hans Zobel, Water Mill, NY (US)

Correspondence Address: HOFFMANN & BARON, LLP, 6900 JERICHO TURNPIKE, SYOSSET, NY, 11791, US

(73) Assignee: ZIEHM MEDICAL LLC, Hauppauge, NY (US)

(21) Appl. No.: 12/324,211

(22) Filed: Nov. 26, 2008

Related U.S. Application Data
(60) Provisional application No. 60/991,505, filed on Nov. 30, 2007.

(51) Int. Cl. A61B 5/00

(52) U.S. Cl. 600/301

(57) ABSTRACT

An apparatus and method for measuring the key elements of human primary health is disclosed. The apparatus is in the form of a medical diagnostics unit capable of measuring Electrocardiogram (ECG), height, weight, body mass index (BMI), body temperature, hearing efficiency, lung function, pulse, blood oxygen levels, blood pressure, urology and vision testing. The medical diagnostics unit includes an enclosure with a data card and/or fingerprint entry. The enclosure includes medical measuring devices which allow a patient to follow instructions on a touch screen visual display unit to conduct the desired tests and obtain the patient's health information. This information is stored locally as well as being transmitted to a doctor for review and evaluation. The automated medical diagnostics unit reduces the staffing requirements to obtain a patient's basic health information.

### CROSS-REFERENCE TO RELATED APPLICATION

The present application claims priority to U.S. Provisional Patent Application Ser. No. 60/991,505 filed Nov. 30, 2007, the disclosure of which is incorporated herein by reference in its entirety.

### FIELD OF THE INVENTION

The present invention relates to an automated medical information gathering apparatus to obtain a patient's basic health information.

### BACKGROUND OF THE INVENTION

Conventionally, when a patient desires to have a medical examination, a nurse conducts various tests to obtain a patient's basic health information. These tests typically include various measurements such as height, weight, blood pressure, blood oxygen, body temperature, an electrocardiogram, vision, hearing, lung function, and urology testing, and so on. The measured data is manually entered on to a chart or into a computer system, which data entry is slow and prone to error. Accordingly, it would be beneficial to the health care industry to have an apparatus that allows the patient to self-administer basic medical measurements to obtain health information which is directly and accurately stored on a computerized system for evaluation and analysis by a health care provider. The apparatus would reduce unnecessary staffing, speed the process to obtain a patient's health information and provide repeatable accurate health data to a doctor for evaluation.

### SUMMARY OF THE INVENTION

According to aspects illustrated herein, a medical diagnostics apparatus is disclosed that includes a portable enclosure, medical test units, a video display, and a host computer. The portable enclosure includes an access door dimensioned to permit a person to enter the enclosure and forms an interior area enclosed by a platform and walls to promote patient privacy. The medical test units are used to conduct health-related self-test measurements and the video display unit, which includes a touch-screen, provides instructions to a patient to operate the medical test units. The host computer receives and stores results transmitted from the medical test units. The medical test units can be used to determine a patient's height, weight, pulse, blood oxygen level, blood pressure, and lung capacity.

In some embodiments, the medical test units can include a blood pressure unit, an electrocardiogram (ECG) unit, blood analysis unit, and a urine analysis unit. The blood pressure unit can include a sphygmomanometer that is movably mounted on a track, where the sphygmomanometer is configured to slide along the track fixed on a shelf in the enclosure. The electrocardiogram (ECG) unit can include transducers pivotally coupled to an underside of a chair in the enclosure via arms, where the transducers can be configured to be positioned about a seat of the chair. The arms of the ECG unit can be telescopically configured so that the transducers can be pushed away from the seat of the chair. The ECG unit can also include transducers operatively connected to a platform of the enclosure for receiving a patient's feet during an ECG test.

In some embodiments, a vending unit can be integrated with the medical diagnostics unit. The vending unit can be configured to vend a sealed container of items required for performance of health-related self test measurements associated with the medical diagnostics apparatus.

In some embodiments, the enclosure can be formed from body sections and ceiling sections. The body sections and the ceiling sections can be dimensioned to fit through a doorway of an existing structure. The body sections and the ceiling sections can be assembled within the existing structure and can have an assembled dimension that is larger than the doorway.

According to other aspect illustrated herein, a medical diagnostics system is disclosed that includes a medical diagnostics unit, and a remote computer. The medical diagnostics unit is formed from a portable enclosure having an interior area defined by walls and an access door dimensioned to permit a person to enter the enclosure. The medical diagnostics unit includes medical test units for conducting health-related self-test measurements, a video display unit comprising a touch-screen for providing instructions to a patient to operate the medical test units, and a host computer adapted to receive and store results transmitted from the medical test units. The remote computer is configured to access the results of the health-related self-test measurements using a web-based medical management application. In some embodiments, the remote computer can be configured to communicate with the host computer using the web-based medical management application to access the results.

The medical diagnostics system can also include a data storage system and/or a server. The data storage system can receive and store the results from the host computer, and the remote computer can be configured to communicate with the data storage system using the web-based medical management application to access the results. The server can be configured to provide the web-based medical management application to the remote computer. Access to the web-based medical management application and portions thereof can be restricted to authorized users. Access to portions of the web-based medical management application can also be restricted based on roles of the authorized users.

According to other aspect illustrated herein, a method of conducting and recording health information of patient is disclosed. The method includes providing a medical diagnostics unit that includes medical test units for conducting health-related measurements mounted within an enclosure adapted to permit a patient to enter the unit. The method also includes testing the patient by conducting a plurality of health-related self-test measurements using the medical test units, recording patient information obtained by use of the medical test units in a host computer located within the medical diagnostics unit and viewing the results of the testing by an authorized user at a location remote to the medical diagnostics unit. In some embodiments, the method can also include restricting viewing the results based on a role of the authorized user and/or restricting access to the medical diagnostics unit to patients having a computer readable personal identification (ID) card, where the patient ID card can be read by the medical diagnostics unit.

The medical diagnostics unit is preferably transportable, provides fully automated data gathering, a computer-based result storage, transmission of the data via Ethernet cabling to other remote computers, automated calibration of all medical measurements devices, unassisted operation by the patient and ease of system use via an integrated touch-screen visual display unit (VDU).

Typically, in medical centers where primary health checks are carried out, health information measurements such as height, weight, body temperature, pulse, blood oxygen, lung capacity testing, etc. are usually executed by a member of the center's nursing staff. The present invention eliminates the need for separate staffing of the primary health checks for patients. Instead, the patient enters the enclosure formed in accordance with the present invention and, having identified themselves to the system via a card reader, is automatically guided through a pre-determined set of sequential medical tests to obtain the desired medical information.

The patient's test results are preferably stored in the host computer and are made available for the patient's medical professional to review either in real-time or at a later date. The patient does not necessarily have to wait for further instructions from the medical center staff before departing from the medical center. The patient's medical professional will have the opportunity to examine the patient's test results and, if required, change the type of tests for the next system session.

### BRIEF DESCRIPTION OF THE DRAWINGS

FIG. 1A illustrates medical diagnostics units arranged for use in a medical center;

FIG. 1B illustrates medical diagnostics units arranged for use by military personnel;

FIG. 1C illustrates medical diagnostics units arranged for use to conduct mass patient screening;

FIG. 1D illustrates medical diagnostics units arranged for self-service use by patients in a retail outlet;

FIG. 2 is a block diagram of an architecture of a system in which one or more medical diagnostics units can be implemented;

FIG. 3A-B illustrates a closed and open view of an embodiment of a medical diagnostics unit formed in accordance with the present invention;

FIG. 4 is an enlarged view of an embodiment of a pulse and oxygen clamp;

FIG. 5 is an enlarged view of an embodiment of the lung test apparatus;

FIG. 6 illustrates an electrocardiogram (ECG) belt used in embodiments of the medical diagnostics unit;

FIG. 7 is an enlarged view of an embodiment of the optical test unit shown;

FIG. 8A illustrates a disassembled view of another embodiment of a medical diagnostics unit formed in accordance with the present invention;

FIG. 8B illustrates a partial assembly and an interior view of the medical diagnostics unit of FIG. 8A;

FIG. 8C illustrates an assembled view of the medical diagnostics unit of FIG. 8A in a closed position;

FIG. 9 is an exemplary embodiment of a blood pressure unit that can be implemented in the medical diagnostics unit;

FIGS. 10A-B show an exemplary embodiment of an ECG unit that can be implemented in the medical diagnostics unit;

FIG. 11-16 are exemplary screen shot that illustrate an administration portion of the web-based medical management application;

FIG. 17-19 are exemplary screen shot that illustrate a patient registration portion of the web-based medical management application;

FIGS. 20-22 are exemplary screen shots illustrating a test request/results portion of the web-based management application; and

FIG. 23 is a flow chart illustrating an implementation in accordance with embodiments of the present invention.

### DETAILED DESCRIPTION OF THE PREFERRED EMBODIMENTS

The present invention is an automated medical information gathering apparatus in the form of a medical diagnostics unit. The unit includes an enclosure in which is located medical test units to conduct a series of diagnostic tests and measurements to obtain patient health information. The apparatus is preferably transportable and allows the patient to self administer the tests and measurements by following instructions on a computer provided in the apparatus. The results of the tests and measurements can be input directly to a database allowing the results to be reviewed and analyzed by an authorized health care provider located away from the apparatus.

The automated medical diagnostics unit formed in accordance with the present invention may be used in a variety of different areas of patient health monitoring. For example, the apparatus may be used in a private practice doctor's office, hospital emergency room, military personnel health checks, retail outlets, or ultimately for mass population screening.

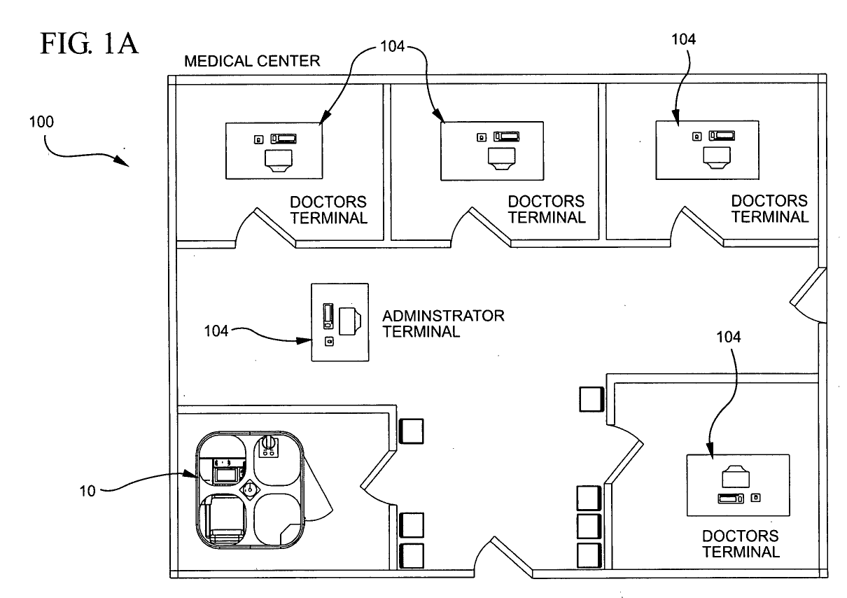

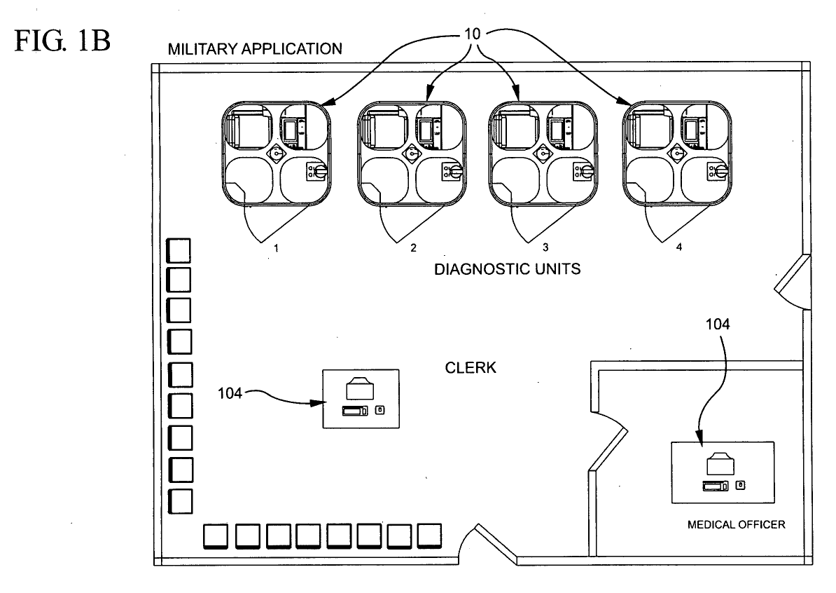

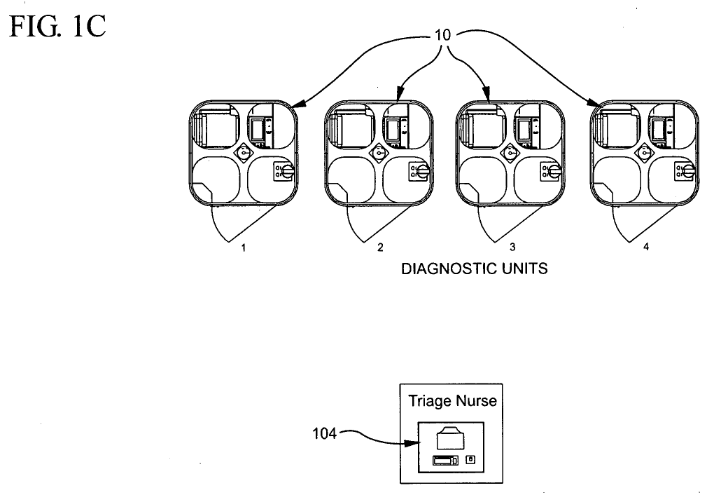

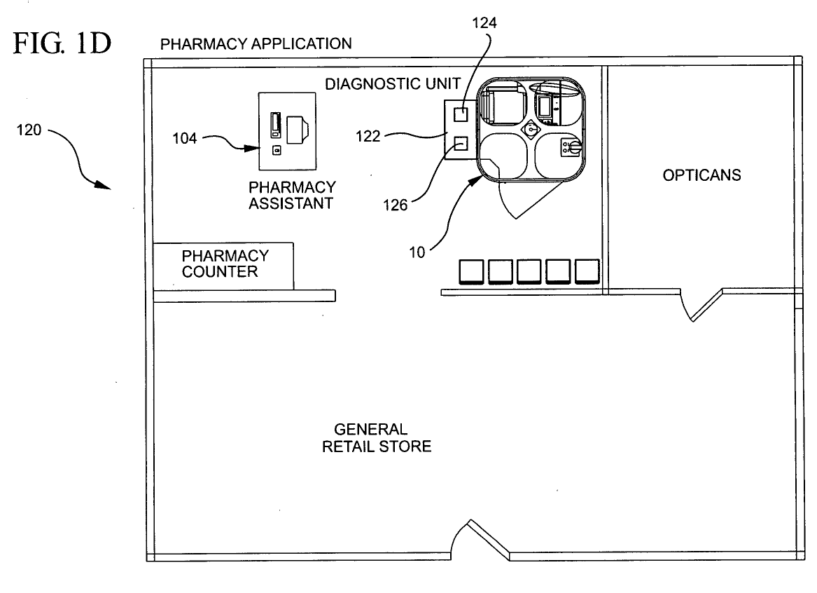

In most medical centers, there is a central administrative person managing patient inquiries and initial registration paperwork. In addition, the medical centers have several general practitioners or specialist providers sharing the facility. As shown in FIG. 1A, a medical center 100, or portion thereof, can have a medical diagnostics unit 10 to provide doctors associated with the medical center 100 with a centralized patient monitoring test unit. The doctors can book their respective patients into the diagnostics unit 10 for completion of required and planned medical tests. The patients can be informed of their appointment time, as well as the number and type of tests to be conducted. The administrative staff of the medical center 100 can also be informed of the appointments, the number and types of tests, as well as the timing and proposed duration of each appointment.

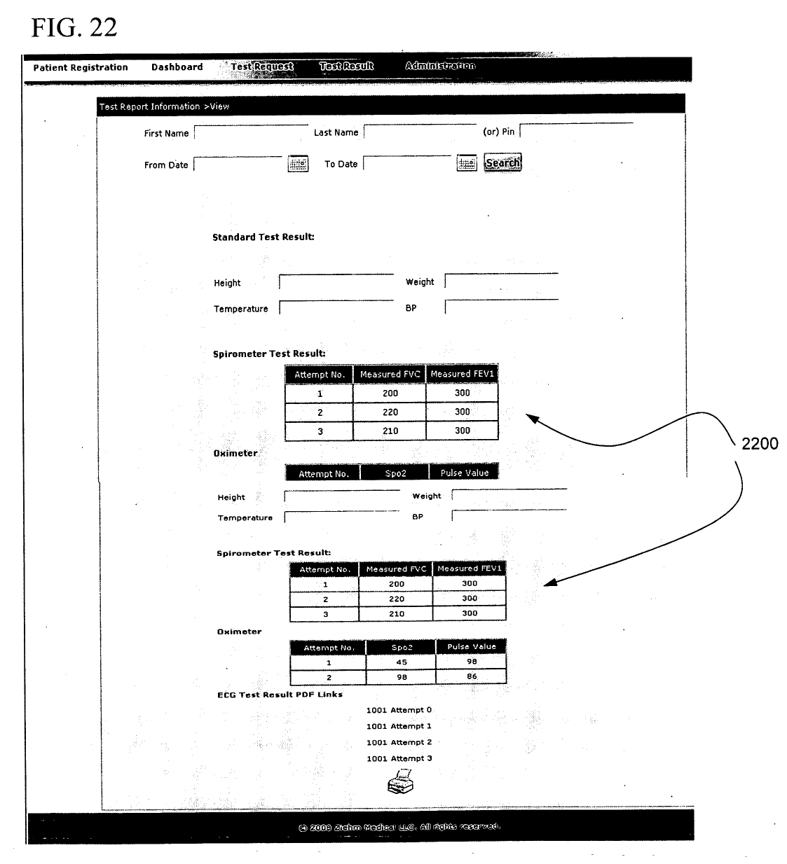

The doctors can have an opportunity to either remotely monitor the test results on a remote computer 104 using a web-based medical management application before the patient leaves the medical center 100 or can examine their patient's test results at a later time for subsequent referral interviews with their patients. (See e.g. FIG. 22). The doctors can access the test results of their own specific patients and preferably not those of other doctors or specialists without specific authorization. A head specialist or medical center manager, who has overall authorized access to the system, may be provided with authority to provide the medical information to other doctors approved by the patient. The patient can review their individual medical test results on the doctor's computer 104, using print outs, or in some embodiments by logging into the web-based medical management application.

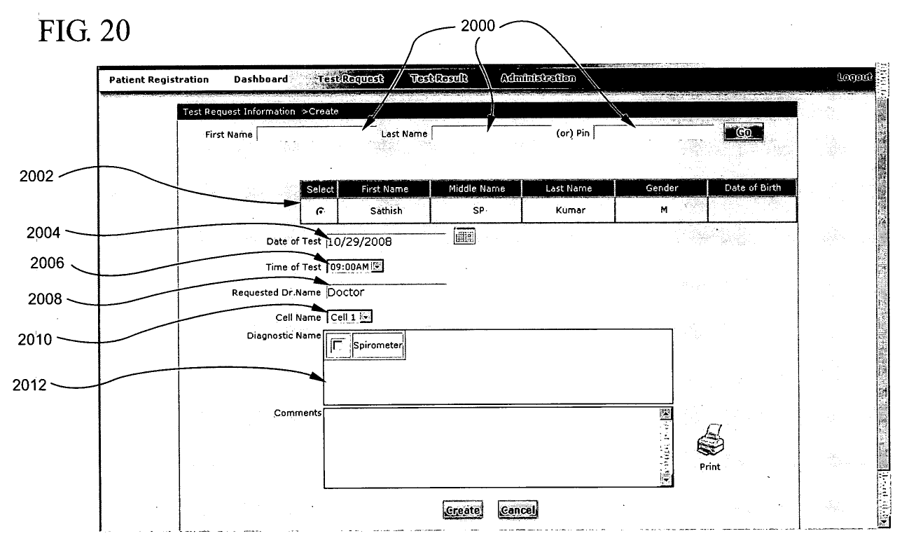

In addition, the doctors can change any or all of the follow-on patient tests directly from their computer 104 and store the updated test schedule information into a diagnostics host computer using the web-based medical management application. (See e.g. FIG. 20). Thus, when the patient uses the medical diagnostics unit 10 at the next planned visit, the medical diagnostics unit 10 automatically aligns the tests to those required by the patient's doctor or specialist. In some embodiments, the medical diagnostics unit 10 can include a card reader to identify the patient via a personal ID card and can match the patient with the tests to be performed as ordered by the doctor. Thus, the patient is efficiently moved through a series of diagnostic tests and measurements and the patient's medical information is directly input into a database to be viewed by the patient's doctor using the web-based medical management application. Alternatively, the information obtained by the diagnostic tests and measurements may be written onto a patient's ID card or smart card. The doctor can access the test results when the patient visits the doctor by reading the results stored on the card using a card reader associated with the doctor's computer.

A further example for use of the medical diagnostics unit 10 is in the area of military personnel management and care in which there exists a new or increased demand for ‘batch’ health screening of troops. This can particularly appropriate for members of the armed forces who are about to depart or return from active duties overseas. The medical officer responsible for military health of groups of military personnel, whether at individual or platoon up to brigade level numbers, could utilize the medical diagnostics unit 10 for accurate and planned batch processing of groups or batches of staff over a short time period.

As shown in FIG. 1B, the process of use of the medical diagnostics unit 10 of the present invention is similar to that described above with respect to use by a medical center 100 shown in FIG. 1A, but can preferably involve several medical diagnostics units 10 working in parallel, each of which can be communicatively coupled to one or more remote computers 104 using well known networking technology including wired and wireless implementations. Each of the medical diagnostics units 10 are self-sufficient, stand-alone devices, but their collective, networked data can be centrally stored on a central data storage system, which can be implemented using one or more computers.

A still further example for use of the medical diagnostics unit 10 of the present invention is following a major social disaster, such as hurricane Katrina, where there is a need for large groups of the affected population to be screened for health-related problems. Doctors and hospitals at this time are overwhelmed with casualties and little medical analysis time can be spent on basic health checks of other, less injured people. An arrangement of medical diagnostics units 10, as shown in FIG. 1C, provides medical personnel with an essential aid for large group screening of primary health indicators, e.g., increased body temperature due to water-borne diseases, increased hypertension from shock or loss of medication and urinary infections. The rugged portability of the medical diagnostics unit 10 makes it ideal for use in such circumstances. The medical diagnostic unit 10 can be transported to the nearest medical triage center 110 in the disaster area and can be provided electrical power from a standard 110 vac or 220 vac generator. The doctors can appoint limited trained staff to monitor the results of critical health measurements from a central location, such as the remote computer 104, using the web-based medical management application and can process patients that require follow-up medication or treatment in response to the test results. The patient's health information can be obtained quickly using the self-administered tests and measurements, the results of which are accurately recorded and transmitted for review and evaluation.

As shown in FIG. 1D, the medical diagnostic unit 10 can be configured for installation in a commercial retail outlet 120, or other commercial location. The medical diagnostic unit 10 can be implemented in such locations with a minimum level of administrative support. The patient can select and pay for their chosen medical tests by utilizing a ticketing and/or vending unit 122, which can dispense ID cards 124 and/or sealed containers 126 that include disposable items required during the testing. For example, the sealed containers 126 can include a lung mouthpiece filter, nose clip, foot coverings, an alcohol wipe, and the like. The vending unit 122 is preferably integrated into the medical diagnostic unit 10, but can be implemented as a separate stand alone unit in some embodiments.

The administrator, such as a cashier, sales assistant, pharmacist, and the like, may assist the patient if there are any questions regarding the tests or operation of the medical diagnostic unit 10. Finally, in some embodiments, the results of the tests are not stored or transmitted to other locations to protect the patient's sensitive information. In such embodiments, the patient can request a copy of the test results from, for example, an integrated printer collocated with the medical diagnostic unit 10. In other embodiments, the patient can be prompted with a list of medical professionals and the user can select one or more of the medical professions to whom the test results are to be sent. The test results can be sent using a secure connection to the selected medical professionals. For example, the patient can choose to send the test results to the patient's primary care physician, who can review the results to identify potential health issues.

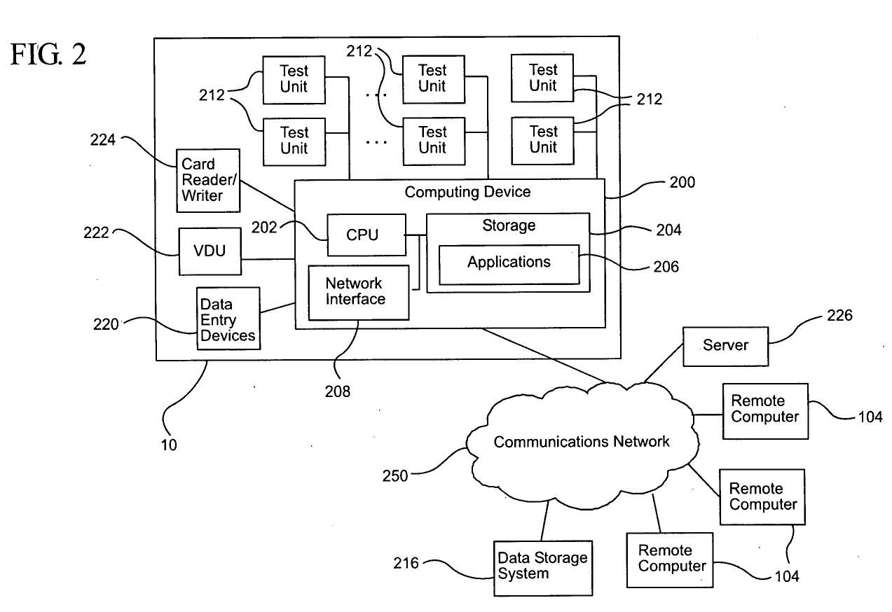

FIG. 2 depicts an illustrative architecture for implementing a system in which the medical diagnostics unit 10 can be use. The medical diagnostics unit 10 includes a host computer 200 communicatively connected to installed medical test devices 212 in the medical diagnostics unit 10 and to remote computers 104. The host computer 200 and the medical test devices 212 can be wirelessly connected or connected using wires, such as Ethernet cables or RS-232 cables, so that the host computer 200 and the medical test units 212 can communicate with each other.

The host computer 200 can be a mainframe, personal computer (PC), laptop computer, workstation, handheld device, such as a PDA, or the like, and preferably is a standard computing device incorporating a central processing unit (CPU) 202 and storage 204. The host computer 200 can receive interface with data entry device(s) 220, such as a keyboard, microphone, and/or mouse, and can interface with a video display unit (VDU) 222 and one or more card readers/writers 224 to receive and output information. The storage 204 can store data, such as patient personal data, patient health data, appointment dates, and the like, as well as instructions, such as instructions for implementing applications to facilitate performance of heath-related tests using the medical test units 212, or to facilitate review and management of patient personal data, patient health data, and/or appointments. The storage 204 can include such technologies as a floppy drive, hard drive, tape drive, Flash drive, optical drive, read only memory (ROM), random access memory (RAM), and the like. The storage 204 can be local or remote to the host computer 200.

Applications 206, such as a medical diagnostics application can be resident in the storage 204. The applications 206 can include instructions for implementing the embodiments of the present invention. The medical diagnostics application can illustrate and instruct patients using a display and or audio speaker on how to perform the tests. The medical diagnostics application can also be configured to receive, format, store, and make available test results of the tests being performed from the test units 212. The CPU 202 operates to run the application in storage 204 by performing instructions therein and storing data resulting from the performed instructions, which may be presented to an operator, such as a patient, Doctor, administrator, and the like, via the remote computer or by other mechanisms known to those skilled in the art, such a print out from a printer 214 associated with the host computer 200.

The host computer 200 includes a network interface 208 to facilitate communication with the test units 212, the remote computers 104, and the data storage system 216. In some embodiments, the host computer 200 stores patient personal data, test results, appointment dates, and the like. In other embodiments, patient personal data, test results, appointment dates, and the like, are stored remotely from the host computer 200 using, for example, a centralized data storage system 216 formed from one or more computing devices. The connection between the host computer 200 and the remote computers 104 can be implemented wirelessly or via physical wires, such as Ethernet cables. The host computer 200 is preferably communicatively connected to remote computers 104, such as ‘Office’ and ‘Doctor's’ computers, via a network 250. The connection between the host computer 200 and the remote computers 104 can be implemented wirelessly or via physical wires, such as Ethernet cables.

The host computer 200 is configured to continuously verify that all of the individual medical test units 212 are operating within pre-set calibration limits of accuracy and performance. If any of the test units 212 is indicated as being out of calibration or malfunctions, the host computer 200 issues a ‘system unavailable’ signal to an ‘Office’ computer (e.g., one of the remote computer 104), which can be monitored by an administrator. An authorized medical technician can be sent to analyze the reason for the failure and after rectification, bring the system back on-line or ‘Available’.

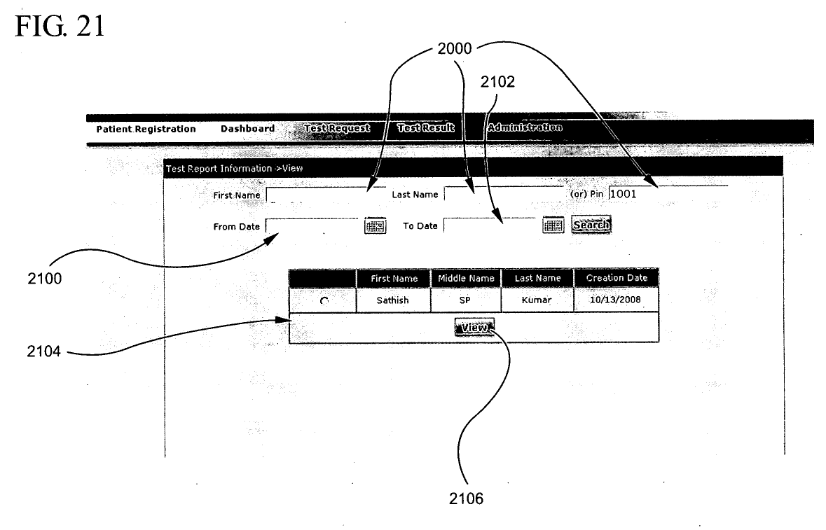

An authorized medical professional may access the patient information data stored on the host computer 200 or a central data storage system 216 using one of the remote computers 104. To achieve this, the medical professional preferably logs into a web-based medical monitoring application, which can be stored on the remote computer 104 or can be provided by a server 226. The medical professional can have several options from a ‘pick’ list menu to obtain the information in a desired format. For example, the physician can request measurement data for a specific patient by the patient's name; the type of test results required; or the date of the taken test. An exemplary screen shot illustrating a patient list is shown in FIG. 21. An exemplary screen shot illustrating an individual's patient information and tests to be conducted is shown in FIG. 20. The accessed results can be viewed on the Doctor's computer for analysis and review and can be downloaded to the Doctor's computer so that the results can be stored on the Doctor's computer.

The doctor or medical specialist may choose to modify the patient's next appointment schedule including repeating each of the tests or selecting alternative tests at the patient's next visit. This may be accomplished by the doctor selecting or deselecting relevant tests from a menu list and sending the request to the host computer 200. An exemplary screen shot illustrating the menu option list of tests to be performed is shown in FIG. 20. The host computer 200 can automatically update the tests to be performed to comply with the doctor's request. At the commencement of the patient's next visit, the medical diagnostics unit 10 can automatically initialize the appropriate tests based the identity of the patient which can be obtained upon reading an ID card associated with the patient when the card swiped into the system's reader inside the enclosure by the patient. An exemplary screen shot of a patient's test results to be reviewed and analyzed by an authorized medial professional is shown in FIG. 22.

As discussed above, the Office computer (e.g., one or the remote computers 104) can be linked (communicatively coupled) to the host computer 200 for use by the medical administrative staff for patient data input. The administrative person can manually enter basic patient data, such as name, address, age, social security number, insurance information, medications, allergies, the patient's photograph, etc., using the web-based medical management application. This data entry is used during the first visit to the medical facility and thereafter if the patient's personal data changes.

The individual medical test units 212, included in the medical diagnostics unit 10, obtain the medical measurement information of the patient. In a preferred embodiment, the test units 212 are stand-alone devices that can be located on shelves, within equipment cabinets, or in the case of, for example, a scale, within an enclosure of the medical diagnostics unit 10 for access by the patient. The test units 212 are preferably linked to the host computer 200 by a wireless connection or by RS232C cables, and each test unit 212 can receive its own 110 vac power line input, 220 vac power line input, or other suitable power inputs. In some embodiments, the test units 212 may be known devices that can obtain the desired patient information, and all such devices are contemplated to fall within the scope of the invention.

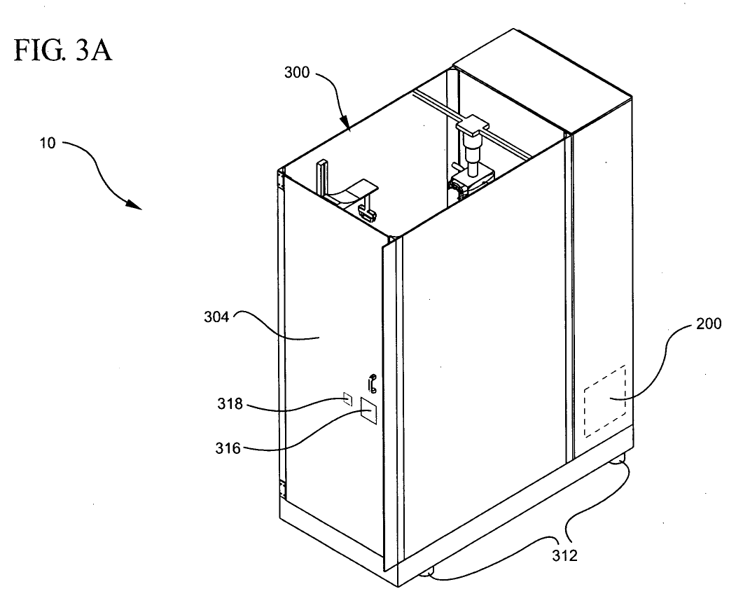

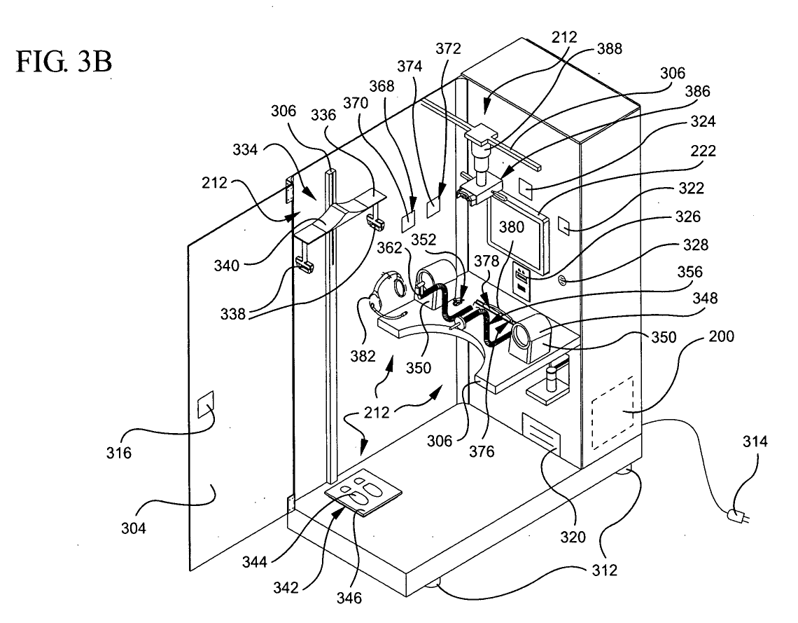

As shown in FIG. 3, one embodiment of the medical diagnostics unit 10 has an enclosure 300 in the form of a single occupancy booth containing either a built-in seat or chair provided therein, the medical test units 212, a touch-screen video display unit (VDU) 222 and an access door 304. The enclosure 300 preferably comprises a metal frame with infill opaque side panels and equipment consoles 306. The equipment consoles 306 contain the appropriate medical test units for the health check measurements. For example, the equipment console 306 includes the host computer 200 communicatively coupled, wirelessly or via physical wired connections, to all medical test units 212, and to all relevant remote desktop or laptop computers 104, as shown in FIGS. 1A-1D and 2.

In a preferred embodiment, the medical diagnostics unit 10 is mobile and includes at least four lockable wheels 312 mounted to the bottom of the enclosure 300. The medical diagnostics unit 10 receives power via an earthed power cord 314 for connection to a local 110 vac standard supply socket, a 220 vac standard supply socket, or other power sources suitable for supplying the medical diagnostic unit 10 with sufficient power. The equipment consoles 306 are preferably accessible from the external facets of the enclosure 300 via lockable cabinet doors to facilitate medical or administrative personnel to repair, replace, or maintain the test units 212 and/or components of the medical diagnostics unit 10.

The access door 304 and side panels of the enclosure 300 are preferably made of opaque plastic to allow external ambient light to enter the enclosure and to provide patient privacy. Additional lighting may be provided inside the enclosure 300 for patient comfort and ease of system use. The enclosure access door 304 includes a lock mechanism 316. The access door 304 automatically locks to prevent unauthorized access during the diagnostic testing procedures. The access door 304 is normally locked and may be opened by a patient through the use of a personal ID card via a barcode reader or smart card reader door lock 318. When the patient swipes their personal ID card near the door lock reader 318, the access door 304 unlocks and is available for the patient to open for entry into the booth. In the event of an emergency, the patient can exit the enclosure 300 by operating an internal door handle, which bypasses the door lock mechanism 316 allowing the access door 304 to be opened. In order to maximize hygiene levels, the enclosure 300 minimizes internal corners and crevices inside the enclosure 300 and can include an ultra-violet air purifier unit 320, which can be used to purify air entering, and within, the enclosure 300. Furthermore, the plastic panels forming the enclosure may be formed to include a bactericide or antimicrobial agent in the plastic, such as, for example, Microban®. In addition, the internal surfaces of the enclosure 300 are capable of withstanding frequent cleaning by a pre-treated ‘wet-wipe’.

The color touch-screen visual display unit (VDU) 222, as well as one or more loudspeakers 322 and microphones 324, are preferably mounted on a wall formed by one of the side panels. The microphone 324 can be provided to facilitate voice recognition communication between a patient and the medical diagnostic unit 10. The touch screen VDU 222 and/or the microphone can enable patients to access the various medical test prompt screens of the health check diagnostic application on the VDU 222.

A card reader 326 can be mounted to a wall on the interior of the enclosure 300, which allows a patient to gain access to the health check diagnostics application loaded on the host computer 200 by swiping the personal ID card. The medical diagnostics unit 10 can also include a biometric reader 328, which can be a fingerprint reader that can scan a patient's fingerprint when the patient places a finger digit on the local fingerprint reader. The scanned fingerprint can be compared with that of a fingerprint stored remotely to determine whether the finger prints match before the test procedure begins.

The interior of the medical diagnostics unit 10 can include equipment consoles 330 for implementing health tests or screening using the test units 212. For example, the medical diagnostics unit 10 can include a height unit 332, a weight unit 342, blood pressure unit 348, a pulse and blood oxygen level unit 352, a body temperature unit 356, a lung function unit 362, a urine analysis unit 368, a blood analysis unit 372, an electrocardiogram (ECG) unit 376, a hearing measuring unit 382, and a vision acuity unit 386.

The height unit 332 can include a height gauge 334 having a horizontal bar 336 with associated grab handles 338 to be used when determining the height of a patient. The patient can stand in front of the height gauge 334 and upon prompting by the VDU 222, the patient can grip the grab handles 338 and pull the horizontal bar 336 downward until a height gauge plate 340 that is attached to the horizontal bar 336 contacts the top of the patient's cranium. An integrated micro switch in the height gauge plate 340 is then activated to record the position of the plate and height gauge 334. The VDU 222 confirms that the test is concluded and that the height measurement has been stored in patient data files on the host computer 200. When the patient releases the grab handles 338, the horizontal bar 336 automatically rises, by counter weights, to a rest position ready for use by the next patient. Other arrangements for measuring height known to those skilled in the art may be used to obtain this information.

The weight unit 342 can include outlined markers 344 and a scale 346 mounted on the floor of the enclosure 300. The VDU 222 can conduct a weight measurement by instructing the patient to stand upright with their feet on the outlined marker 344 associated with the floor mounted weight scale 346. When the patient is in position and still, the scale 346 can automatically measure the patient's weight. Alternatively, the patient may be instructed via the VDU on-screen prompts and audio messaging to press a start button located adjacent box to the scale 346. The patient can be informed by the VDU 222 and audio messaging that the weight test is completed and that the patient may proceed to the next sequential test or exit the session whichever is appropriate.

Based upon the combined measurement results for height and weight, a body mass index (BMI) may be calculated by the diagnostics program on the host computer, which calculates a patient's BMI and stores the data in the patient profile for review and evaluation by the patient's doctor.

The blood pressure unit 348 can include a blood pressure measuring unit, which may be implemented as an inflatable cuff 350 for measuring systolic and diastolic pressures. The VDU 222 can conduct the blood pressure measurement by instructing the patient to sit on a chair, which can be provided in the medical diagnostics unit 10, and to follow the visual and/or audio prompts by the system. The patient inserts their arm into and through the cuff 350 of the automated blood pressure unit 348 located on a shelf unit within the enclosure 300. The patient initiates the test by touching the ‘start’ button on the VDU 222. The application of external arterial pressure is fully automated and the pressure profile will mimic that normally generated by a doctor's manual blood pressure test. If, at any time, the patient wishes to stop the test, they are able to press a ‘release’ button on top of the blood pressure unit and the system test is terminated.

Following a successful measurement of the patient's diastolic and systolic blood pressure readings, the automatic cuff 350 fully releases the pressure on the patient's arm and the patient can be instructed by the VDU 222 to remove their arm from the blood pressure unit 348. The blood pressure results are stored in patient data files on the host computer 200 and are made available for the patient's doctor to review and analyze.

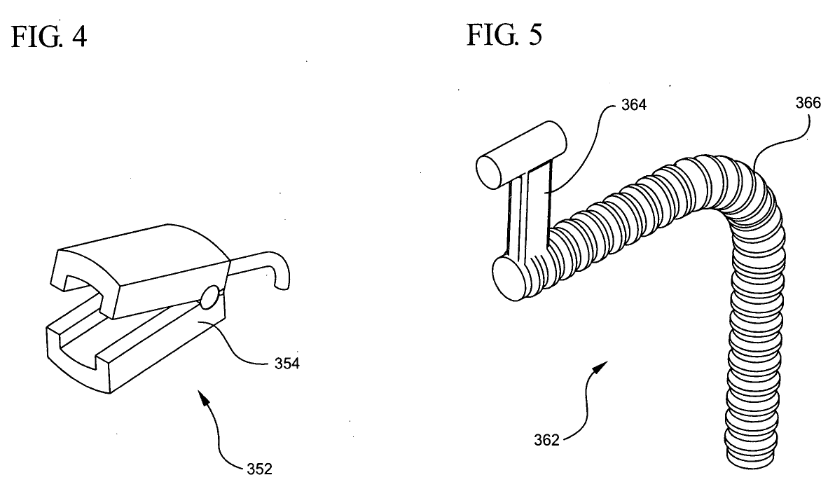

The pulse and blood oxygen unit 352 can measure the pulse and blood oxygen levels using, for example, a standard pulseoximeter finger clamp 354 shown in more detail in FIG. 4. The patient can be prompted by the VDU 222 and audio messaging to place an index finger into a pulseoximeter clamp 354 located on a shelf unit in the enclosure 300. A larger view of the clamp 354 is shown in FIG. 4. The clamp 354 measures the frequency of blood pulsing through the patient's finger and records this blood beat as a heart rate pulse. In addition, the finger clamp 354 is able to display the amount of oxygen in the blood flow. Both readings are stored in the individual patient's medical file on the system's host computer 200.

The body temperature unit 356 can be configured to measure a patient's body temperature and can include a temperature thermocouple and/or an infrared thermometer. For embodiments that include the temperature thermocouple, the temperature thermocouple is attached to a plastic-coated flexible arm. A temperature sensor arm of the temperature thermocouple can be positioned by the patient so that the sensor tip is near to the patient's mouth. The patient, following instructions from the VDU 222 and audio prompts, can cover the temperature sensor arm with a plastic disposable cap from a dispenser in the enclosure 300 so that the disposable cap is secured over the end of the temperature sensor arm. The patient is instructed to close their lips over the disposable cap and touch the VDU ‘start’ button. Following a successful body temperature measurement, the patient is instructed to remove the sensor from their mouth and to remove the disposable cap from the sensor tip and place the cap in a provided medical waste disposal chute.

For embodiments that include the infra-red thermometer 30, a flexible arm is provided with an infra-red thermocouple transducer, which can be placed near the patient's mouth, ear canal, and/or temple to detect infra-red radiation radiating from the patient. When the infra-red thermometer is used to measure the patient's temperature from the patient's mouth, the patient is instructed to open their mouth in front of the sensor, but not contact the sensor, and press the ‘start’ button on the VDU 222.

The thermocouple transducer sensor is preferably a Cadmium Mercury Tellurium sensor that works based on the heat energy radiated from a region of the patient's body (e.g., mouth) and the thermal image result is recorded as the patient's body temperature. Thus, there is no need for the patient to either insert the transducer into their mouth or apply a hygiene cap to the sensor.

Using the body temperature unit 356, the patient's temperature is recorded and stored on the host computer 200. Other methods known to those skilled in the art can be used to obtain the patient's body temperature. The patient's doctor, as with all other measurements, can review a history of previous results as a graph or list.

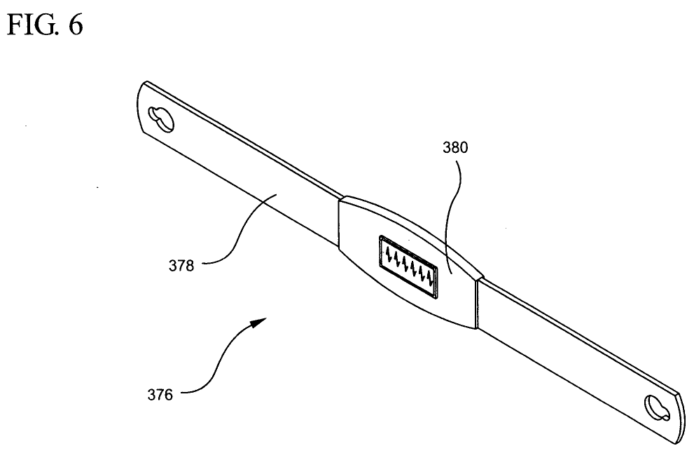

The lung function unit 362 can be implemented in the form of an air velocity transducer 364 attached to a flexible hose 366 near to the face of the seated patient in the enclosure 300. An enlarged view of one embodiment of the lung function unit 362 is shown in FIG. 5. The patient is instructed by the VDU 222 and audio messaging to place a disposable cover over the end of the flexible hose 366 and place the hose 366 into their mouth and apply a nose clip. The nose clip may be attached by a tether to the air velocity transducer. If desired, a disposable cover issued for placement over the nose clamp by the patient prior to commencement of the lung function test can be provided. In a preferred embodiment, the nose clip and mouthpiece filter are preferably disposable items that are discarded by the patient at the conclusion of the lung function test.

The lung function test provides information related to lung capacity and lung expiration rate, i.e., the rate of exhaled air. With respect to testing lung capacity, the patient is instructed by the VDU 222 and audio messaging to place the covered end of the sensor tube into their mouth, apply the nose clip and initiate the test by pressing a “start” button on the VDU 222. This test requires the patient to commence breathing in when prompted for as long as possible. The result of the time of air flow relates to lung capacity.

When the patient is ready, a lung expiration rate test can be implemented using the lung function unit 362. This requires the patient to re-insert the covered end of the sensor tube 364 into their mouth, apply the nose clip and, when prompted, commence breathing out or exhaling as hard as possible. The time of the expired air in the lungs is used to calculate the patient's lung expiration rate. The patient will be informed of the completion of the lung tests and requested to dispose of all sensor caps and nose clip covers into the medical waste chute provided.

The urine analysis unit 368 can be provided for performance of a urology test. The patient can provide a urine sample prior to commencing the diagnostics routine. For example, the patient can be provided a clear plastic container that has a plastic screw top cap. Shortly before the patient enters the medical diagnostics unit 10 for their appropriate appointment, the patient can urinate into the container and apply the screw cap. When the test program is initiated, the patient is instructed to place their urine sample into a tray or cavity 370 on a front panel in the medical diagnostics unit 10 and press a test start button on the VDU 222. In a preferred embodiment, the urology test is fully automated whereby the sample is secured inside the urine analysis unit 368. A sensor, that is capable of measuring primary health indicators within a urine sample, is automatically inserted into the sample container through the closed screw cap to conduct the test. At the end of the test, the sensor is withdrawn from the urine sample and container. The sensor is automatically moved to a cleaning station to be hygienically purged and flushed. The container is automatically transferred to a disposal medical waste chute. The medical waste chutes will be emptied by the medical staff as required. The test results are recorded on the host computer 200 within the medical diagnostics unit 10 as with all previous test results.

The blood analysis unit 372 can be provided for performance of one or more blood tests. The patient can provide a blood sample prior to commencing the diagnostics routine. For example, an administrator, such as a nurse can draw blood from the patient. When the test program is initiated, the patient is instructed to place their blood sample into a tray or cavity 374 on a front panel in the medical diagnostics unit 10 and press a test start button on the VDU 222. In a preferred embodiment, the blood tests are fully automated whereby the sample is secured inside the blood analysis unit 372. The blood analysis unit 372 is capable of measuring primary health indicators within the blood sample. The blood sample is automatically transferred to a disposal medical waste chute. The medical waste chute can be emptied by the medical staff as required. The test results are recorded on the host computer 200 within the unit as with all previous test results.

The medical diagnostics unit 10 is also capable of measuring a patient's electrocardiogram (ECG) using the ECG unit 376. In some embodiments, an ECG transducer that is either hard-wired to the ECG unit 376 or has wireless connection to the same measurement unit 376 can be provided. In some embodiments, a transducer belt 378 (a larger view is shown in FIG. 6) having transducers 380 can be provided to measure record ECG measurements, and in other embodiments transducers on which the patients palms and feet can be placed can be provided as discussed in more detail below.

For embodiments that include a transducer belt 378, the patient, when prompted, removes a transducer belt 378 from it's receptacle on the shelf unit and wraps the transducer belt 378 around their bare chest. The VDU 222 may display graphics providing instructions for the correct body position of the ECG transducer belt 378. Once placed on the body, the patient initiates the test and the ECG unit 376 issues a ‘ring’ test signal to the transducer belt 378 to ensure correct placement of the belt 378 on the patient's chest. If correctly placed, the ECG test commences and completes. The transducer belt 378 preferably incorporates at least five (5) measurement points, but may include as many as twelve (12) or more measurement points. In some embodiments, the patient may not be able to see the test results and the test results are recorded on the host computer 200 for access by the patient's authorized physician. In other embodiments, the results may be displayed by the VDU 222. As previously noted, the door and walls of the unit are opaque and the door automatically locks to prevent unauthorized entry so that the patient is ensured privacy while conducting each of the tests.

The hearing unit 382 can include earphones 384, which in some embodiments can include an integrated microphone. Preferably, the seated patient, when prompted by the VDU 222 and an audio message, places the earphones 384 onto their head and follows the on-screen VDU instructions. The patient can verbally respond to prompts regarding the audio hearing tests. At the end of the audio hearing test, the patient removes the headphones 384 and replaces them in the allocated location ready for use by the next patient. The patient may be provided with disposable hygienic earphone covers for use during the audio test. At the cessation of the audio tests, these covers can be disposed of into the medical waste chute provided in the unit. Like all test results, the audio test results can be recorded and stored on the host computer 200 for access by authorized medical personnel.

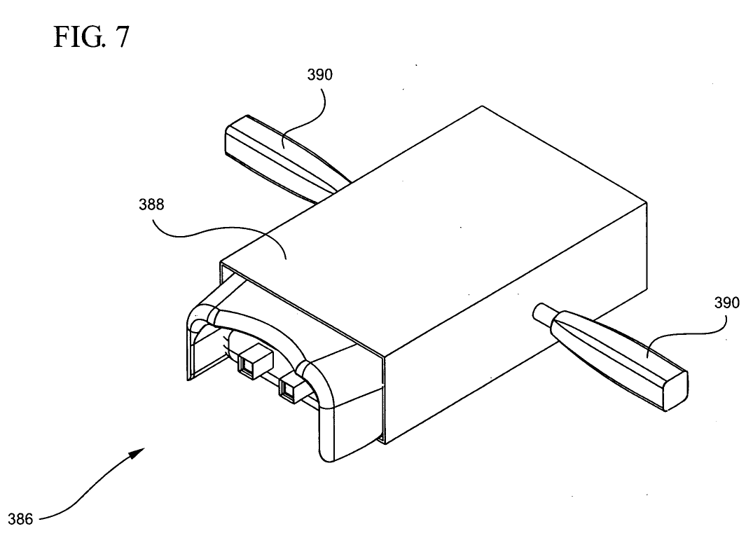

The vision unit 386 can include an optical test unit 388, as shown in FIGS. 2A and 7, which can be attached to a lower end of a counterbalanced ‘periscope’ mechanism 388. The periscope 388 is preferably located ergonomically near the seated patient and preferably includes two handles 390 to enable the patient to pull down and lock the vision unit 386 at a suitable and comfortable height for the patient's use. The vision test unit 386 may also include a chin rest to assist in keeping the patient aligned with the test unit 386. The periscope handles 390 may include two electric micro switches wired in series configuration. When both of the switches are activated by the patient's hand grip, a fail-to-safe solenoid is de-energized and this action disengages a mechanical clutch that enables manual vertical movement of the periscope 388. When the patient releases the handles 390, the periscope 388 will automatically lock in the vertical position. Further vertical travel of the unit can only be possible when the patient again grabs the periscope handles 390.

In a preferred embodiment, the patient can either initiate the vision test by touching the ‘start’ button on the VDU 222 or by pressing an integrated button on the periscope handle 390. The vision unit 386 can incorporate a microphone for verbal responses by the patient during the eye tests. At the completion of the vision test, the patient can be instructed to unlock the periscope 388 and gently raise it to a ‘park’ position. The test results can be recorded and stored on the host computer 200.

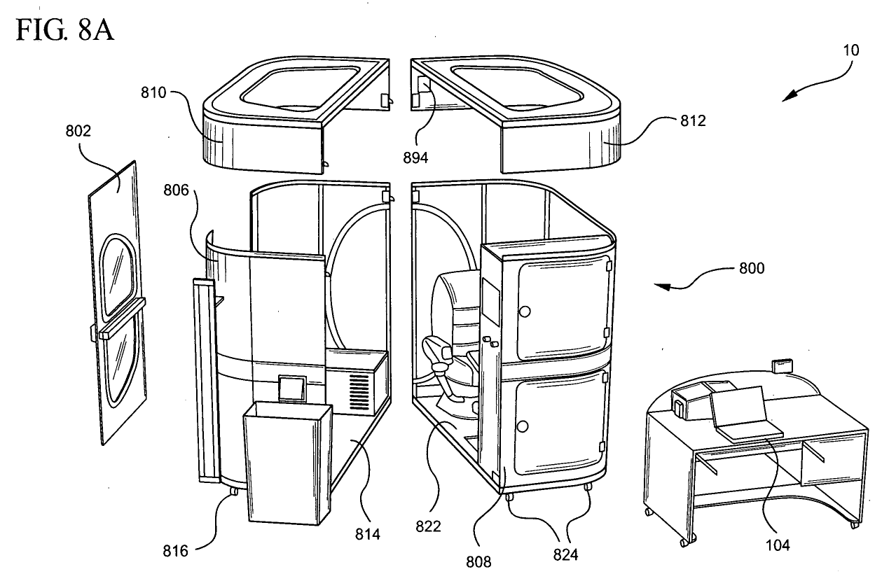

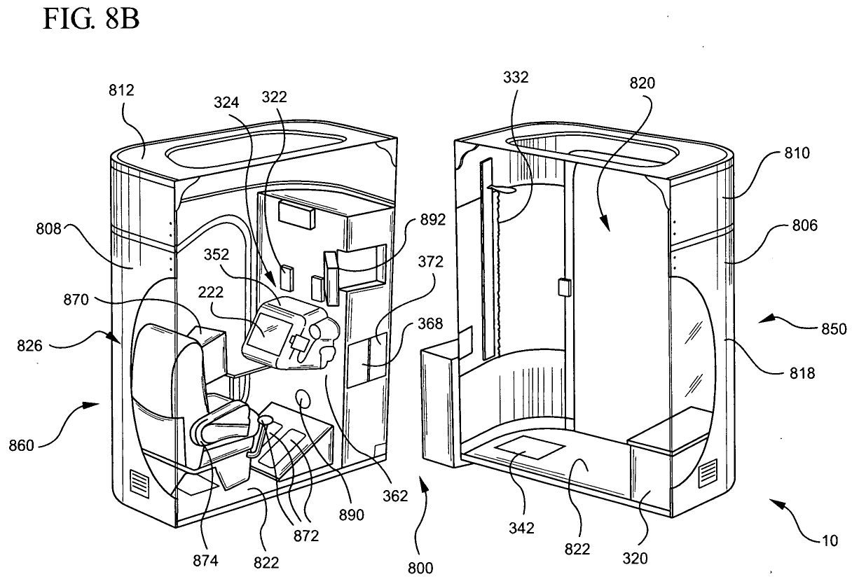

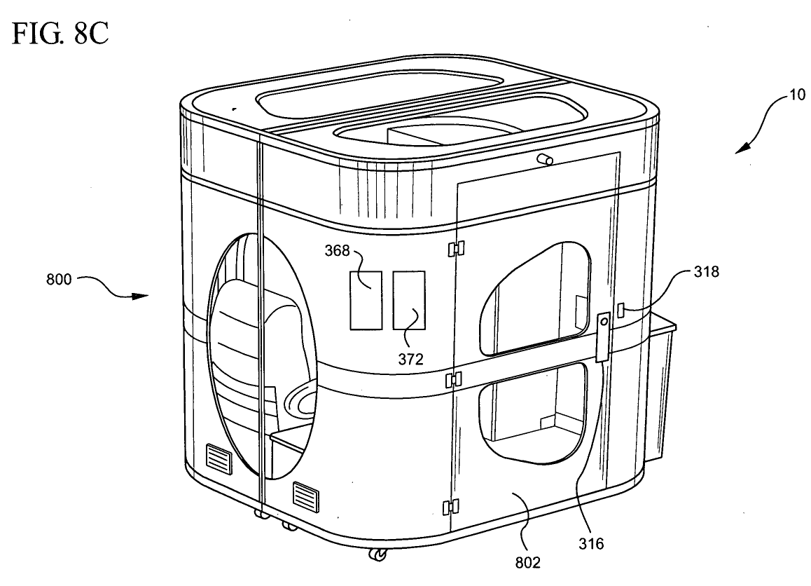

FIGS. 8A-8C illustrate another embodiment of the medical diagnostics unit 10 having an enclosure 800. FIG. 8A shows an exploded perspective view of the enclosure 800 of the medical diagnostics unit 10. FIG. 8B shows the enclosure 800 partially assembled, as well as, an interior area of the medical diagnostics unit 10. FIG. 8C shows an external view of the medical diagnostics unit 10 when the enclosure 800 is fully assembled.

The enclosure 800 is formed from body sections 806 and 808, and ceiling sections 810 and 812. In the medical diagnostics unit's disassembled form, the sections 806, 808, 810, and 812 are dimensioned to fit through doorways and in elevators. For example, the sections, 806, 808, 810, and 812 can dimensioned to fit through a doorway having an opening of about thirty-six (36) inches by about eighty-four (84) inches. This allows the medical diagnostics unit 10 to fit into existing structures where implementation of the medical diagnostics unit 10 would otherwise be prohibitive because of the dimensions of the assembled medical diagnostics unit 10.

The section 806 includes a platform 814 supported by castors 816, which facilitates portability of the section 806. The ceiling section 810 can be secured to a top portion of the section 806 using fastening mechanisms, such as bolts and nuts, screws, latches, and/or other suitable fastening mechanisms. When sections 806 and 810 are attached, an assembled section 850 is formed, where the section 810 forms a ceiling and completes the formation of side walls 818 as well as an access door frame 820 of the section 806. The assembled section 850 can include, for example, the weight unit 342, height measuring unit 332, ultra-violet air purifier 320, and trash receptacle.

The section 808 includes a platform 822 supported by castors 824, which facilitates portability of the section 808. The ceiling section 812 can be secured to a top portion of the section 808 using fastening mechanisms, such as bolts and nuts, screws, latches, and/or other suitable fastening mechanisms. When sections 808 and 812 are attached, an assembled section 860 is formed, where the section 812 forms a ceiling and completes the formation of side walls 826 of the section 808. The assembled section 860 can include, for example, the VDU 222, blood pressure measuring unit 870, electrocardiogram unit 872, electronically adjustable chair 874, the lung function unit 362, the pulse/O2 unit 352, a ventilation fan 890, a video camera 894, speakers 322, and microphone 324, as well as another VDU 892 that is pivotally mounted on a console of the medical diagnostics unit 10, which can be used when the patient's height and weight are being determined. For embodiments that include urine and blood analysis testing, the assembled section 860 can include the urine analysis unit 368 and the blood analysis unit 372.

The electronically adjustable chair 874 allows the patient to adjust the position of the chair 874. For example, the patient can raise or lower the height of the chair 874, move it closer to or further away from the VDU 222. By providing an electronically adjustable chair 874, the medical diagnostics unit 10 can facilitate a comfortable environment for the patient and can ensure that the patient is properly positioned for correct implementation of the health test to be administered using the medical diagnostics unit 10.

To complete assembly of the medical diagnostics unit 10, the assembled sections 850 and 860 can be secured to each other (FIG. 8C) and an access door 802 can be mounted forming an enclosed interior area, which is accessible only through the access door 802. The access door can include the lock mechanism 316 and the card reader 318. The assembled sections 850 and 860 can be secured to each other using fastening mechanisms, such as bolts and nuts, screws, latches, and/or other suitable fastening mechanisms.

In some embodiments, the blood and urine analysis units 368 and 370 can be accessible via an external surface of the medical diagnostics unit 10. In these embodiments, the samples can be taken by an administrator, such as a nurse, and can be placed the in the blood and urine units 368 and 370 when the patient enters the medical diagnostic unit 10 to begin the testing procedure. When analysis of the blood and urine is complete, the samples can be automatically disposed of as medical waste in a container of the medical diagnostics unit 10.

A video camera 894 can be mounted on a wall of the medical diagnostics unit 10. The video camera 894 is normally in the off position and is provided for instances when a patient needs assistance. The camera 894 can be connected to one of the remote computers 104 via a secured connection or closed circuit. When a patient requires assistance, the patient can touch a call assistance button that can be displayed on the VDU 222, at which point the video camera 894 can turn on so that an authorized user can view the interior of the medical diagnostics unit 10. The patient can communicate with the authorized user using the microphone and speakers. When the patient no longer requires assistance, the patient can deselect the call assistance button, which turns the camera 894 off and prevents further interaction between the patient and the authorized user.

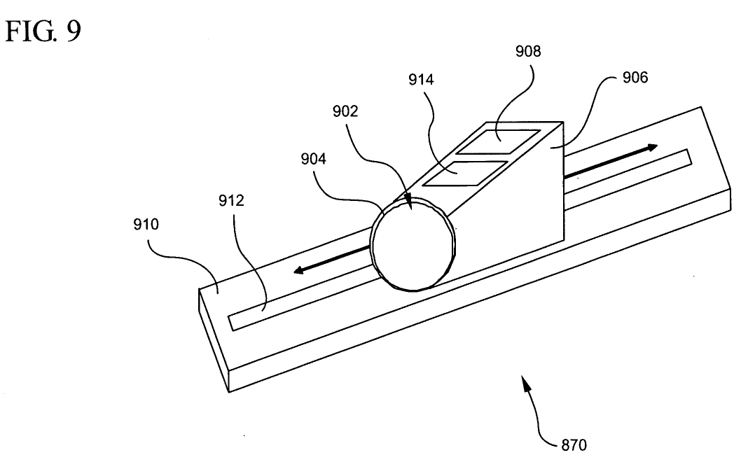

FIG. 9 illustrates an exemplary embodiment of the blood pressure unit 870 that can be implemented in the medical diagnostics unit 10. The blood pressure unit 870 can include a sphygmomanometer 902 having an inflatable cuff 904 contained in a housing 906. The sphygmomanometer 902 analog and/or digital gauges 908 for measuring systolic and diastolic pressure. The sphygmomanometer 902 can be movably coupled to a shelf 910 having a track 912. The position of the sphygmomanometer 902 can be adjusted along the track to allow the sphygmomanometer 902 to be positioned so that the patient's upper arm can extend through the cuff 904 to allow a proper blood pressure reading to be obtained. Accordingly, the patient can electronically adjust the chair and can electronically or manually adjust the position of the sphygmomanometer 902 to achieve a comfortable and appropriate position for implementing the blood pressure test and can allow the patient to slide the sphygmomanometer 902 out of the way when performing other tests so that the sphygmomanometer 902 does not interfere with performance of the other tests. Electronic adjustment of the sphygmomanometer 902 can be facilitated using controls 914 located on the housing 908

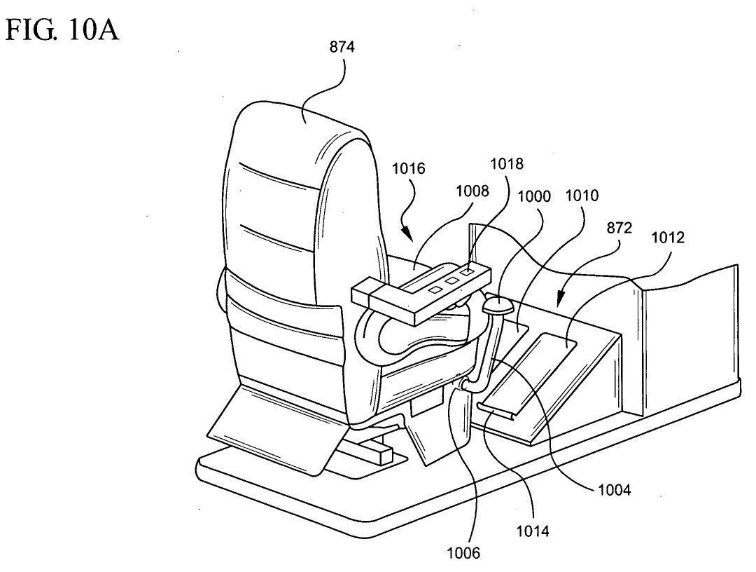

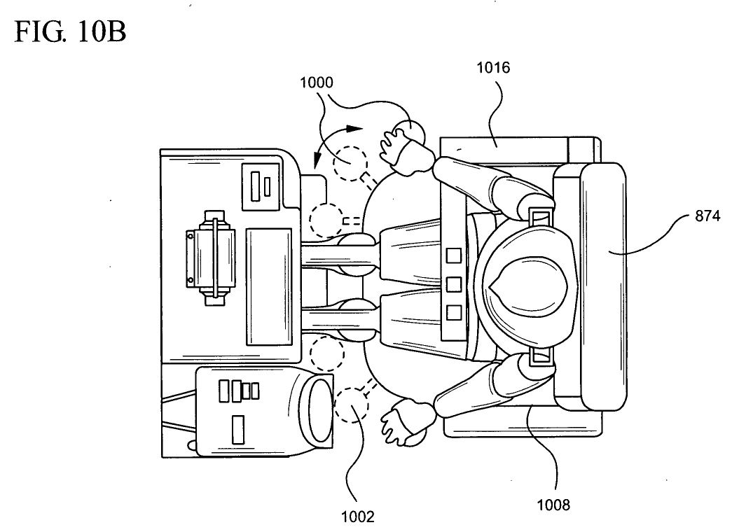

FIGS. 10A and 10B illustrate an exemplary embodiment of the ECG unit 872 that can be implemented in the medical diagnostics unit 10. The ECG unit 872 preferably includes transducers 1000 and 1002, which preferably have a hemispherical or spherical configuration. During the ECG test, the patient rests his/her palms on the transducers 1000 and 1002. The transducers 1000 and 1002 are pivotally and telescopically mounted to an underside of the chair 874 via arms 1004. The arms 1004 can be operatively coupled to the underside of the chair 874 by hinges 1006 such that the arms 1004 can move radially about the hinges 1006 resulting in the transducers 1000 and 1002 being able to move laterally about a seat 1008 of the chair 874. The arms 1004 can also be telescopically configured so that the patient can push the transducers 1000 and 1002 away from the seat of the chair 874 to a position that allows the patient to comfortably rest his/her arms while the ECG test is being performed.

In addition, transducers 1010 and 1012 can be mounted on a base that rests on and/or is coupled to the platform of the medical diagnostics unit 10. The transducers 1010 and 1012 preferably have rectangular configurations with raised lips 1014 at one end. During the ECG test, the patient can rest his/her feet on the transducers 1010 and 1012. The raised lips 1014 can prevent the patient's feet from slipping off the transducers 1010 and 1012 when the ECG test is being performed. The transducers 1000, 1002, 1010, and 1012 can provide four measurement points, which allows the ECG unit 872 to have a six-channel result.

In some embodiments, the ECG unit 872 can include a transducer arm 1016, which can be mounted on a side wall adjacent to the side of the chair 874 or on a side of the chair 874. The transducer arm 1016 can be positioned across, and in contact with, the patient's torso so that transducers 1018 in the arm 1016 are in contact with the patient's torso. The transducer arm 1016 can provide the ECG unit 872 with additional points of measurement, which allows the number of channels for each result to increase.

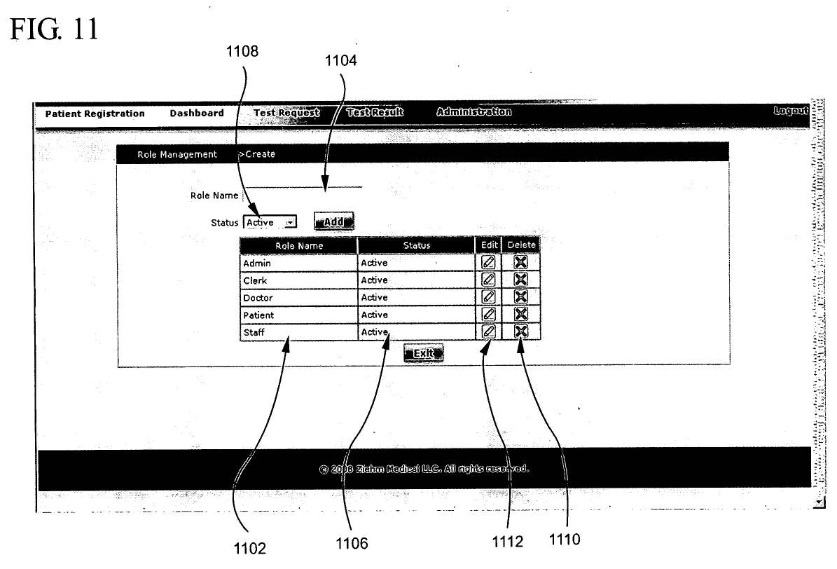

FIG. 11-16 are exemplary screen shots that illustrate an administration portion of the web-based medical management application that can be accessed by the remote computers. Access to the administrative portion of the application can be limited based on the role of the user. For example, administrators can have access to the administration screens, while clerks, doctors, patients, and staff preferably do not have access.

As shown in FIG. 11, roles 1102, such as administrator, clerk, doctor, patient, staff, and the like can be generated by entering a role name in a data entry field 1104. The generated roles can be assigned a status 1106, such as active or inactive using a data entry field 1108. In addition, roll names that have already been created can be deleted using the delete button 1110 or edited using the edit button 1112. The administrator role can be created by default to allow an administrator to set up the various role names and permission.

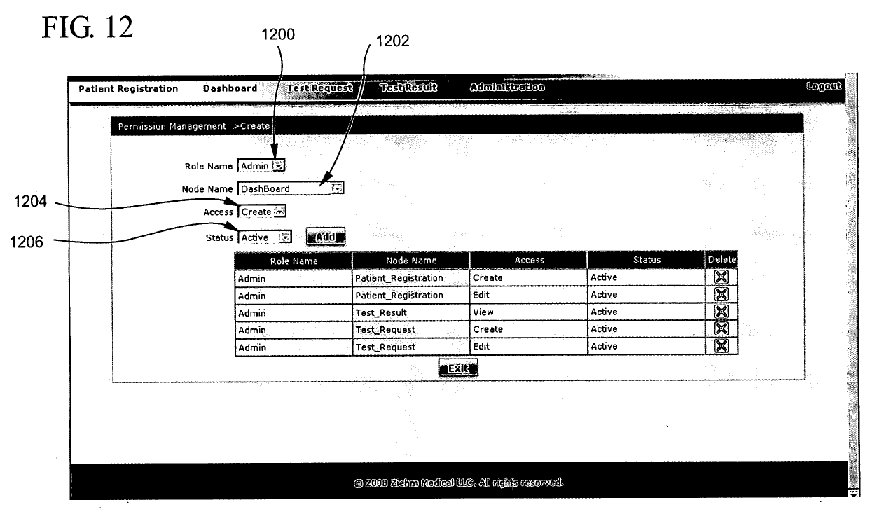

As shown in FIG. 12, permission for accessing various portions of the application can be assigned to the role names. To achieve this, a user, such as the administrator can select a role name using the role name selector 1200, select a portion of the application using the area selector 1202, can select an access or permission level to be associated with the role name for the selected area using the access level selector 1204, and can assign a status for the role name using a data entry field 1206. Access or permission levels can include, for example, view only, edit, create, access denied, and the like.

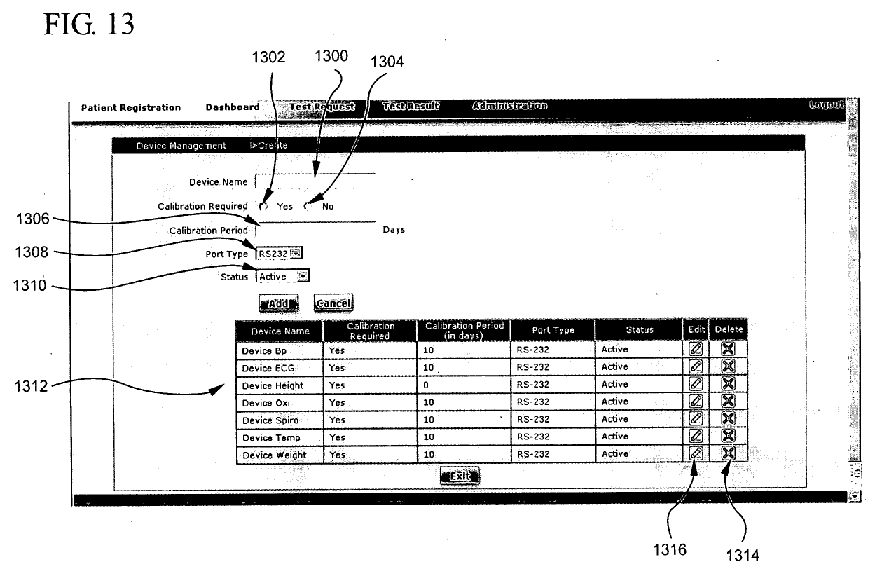

As shown in FIG. 13, an administrator can add device names to the application that are associated with test units that can be implemented in the medical diagnostics unit 10. The administrator can add a device name in a data entry field 1300 and can identify whether the device will need calibration during the device's operation by choosing a ‘yes’ radial button 1302 or ‘no’ radial button 1304. In addition, the administrator can identify the calibration period at which the device must be calibrated using the data entry field 1306. A port type, such as RS-232, can be specified using a port selector 1308, and a status, such as active or inactive, can also be specified using the status selector 1310. A list 1312 of the devices can be provided indicate the administrative parameters and can include a delete button 1314 and an edit button 1316 for each entry in the list 1312.

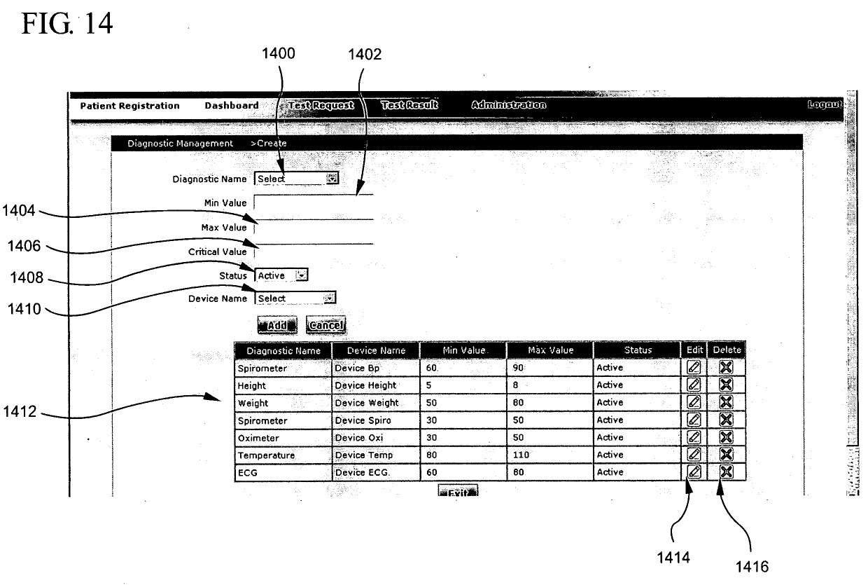

As shown in FIG. 14, diagnostic tests can be added to the application by associating the diagnostic tests with the device name. The administrator can select a name of a diagnostics test using a diagnostic name selector 1400 and can enter minimum, maximum, and critical values for the diagnostic test using data entry fields 1402, 1404, and 1406, respectively. The administrator can select a status for the diagnostic test using the status selector 1408 and can associate the diagnostic test with a device name using the device name selector 1410. A list 1412 can be provided that identifies the diagnostic tests, the device name to which the diagnostic test corresponds, the minimum, maximum, and critical values, and the status of the diagnostic test. Each entry in the list 1412 can be edited using the edit button 1414 or the delete button 1416.

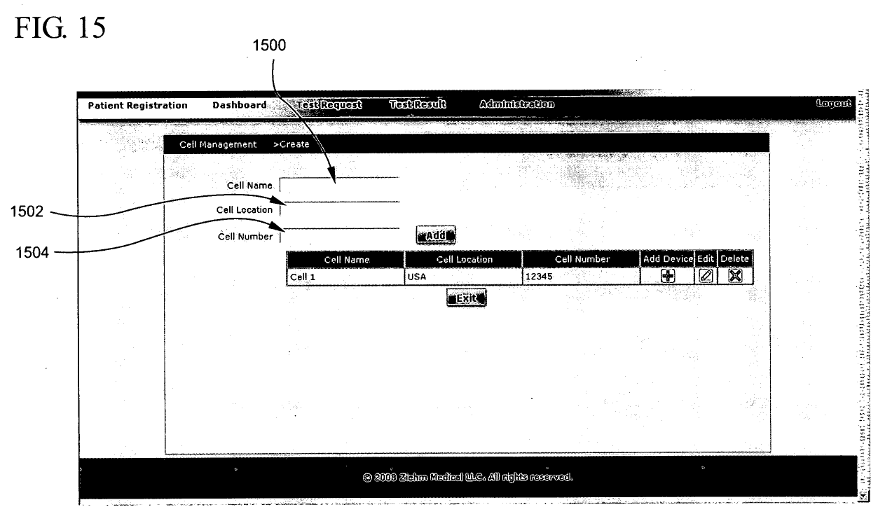

FIG. 15 is an exemplary screen shot illustrating adding medical diagnostic units, which can be referred to as ‘cells’ in the application. The administrator can specify a cell name, cell location, and cell number of the corresponding medical diagnostics unit to be added to the application database using data entry fields 1500, 1502, and 1504, respectively. The cell number can be a unique number assigned to a medical diagnostics unit so that the medical diagnostics unit can be easily distinguished from other medical diagnostics units and can facilitate communication between the application the medical diagnostics unit. Once a medical diagnostics unit has been added to the application database, the administrator can add devices the application database that correspond to test units implemented in the medical diagnostics unit.

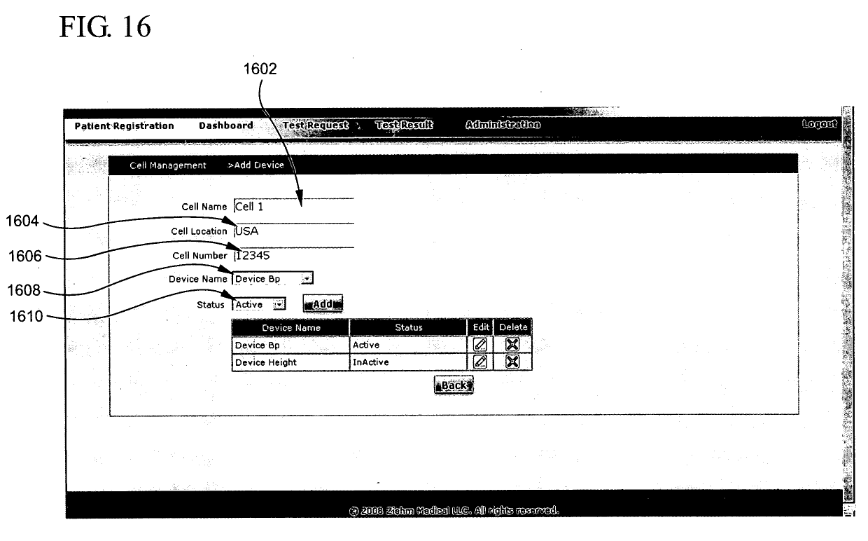

FIG. 16 is an exemplary screen shot illustrating adding devices to the application for a medical diagnostics unit. The administrator enters the cell name, cell location, and cell number using data entry fields 1602, 1604, and 1606. The administrator also selects a device to associate with the medical diagnostics unit using a device name selector 1608. The device names available using the device name selector 1608 can be limited to include only those device names specified by the administrator during the general set up of the application discussed above with reference to FIG. 13. A status selector 1610 can be used to assign a status to the device name.

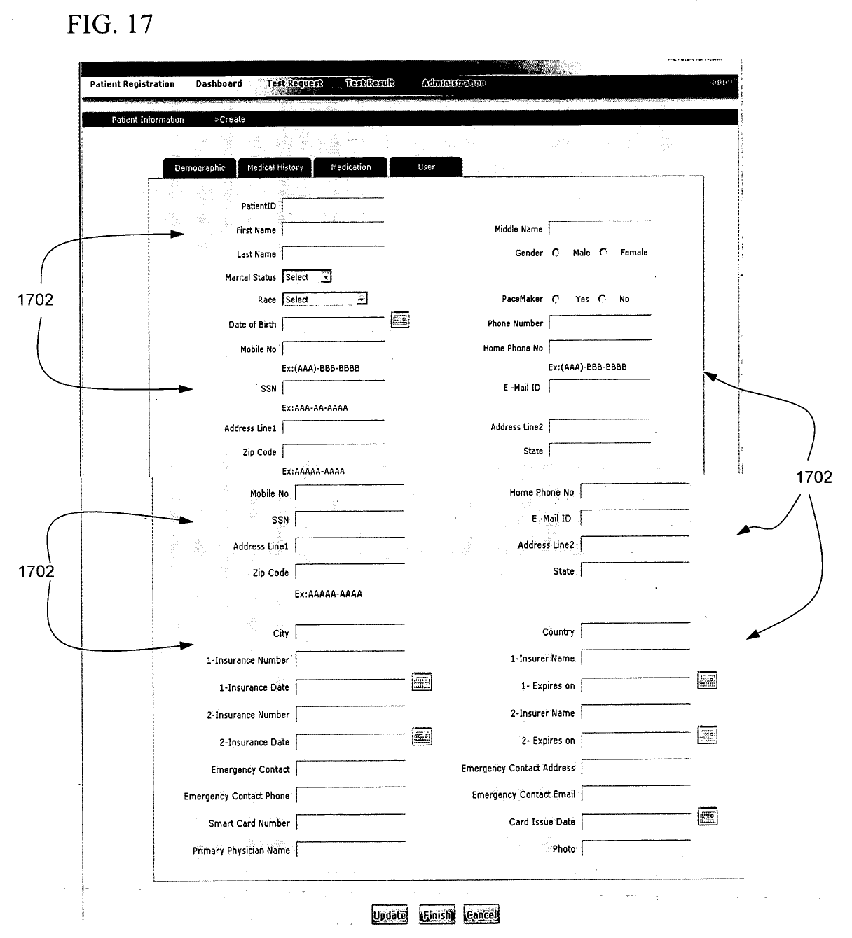

FIG. 17-19 are exemplary screen shots that illustrate a patient registration portion of the web-based medical management application that can be accessed by the remote computers. Access to this portion of the application can be provided to the administrator, a doctor, a clerk, and/or patient to allow background information about the patient to be entered.

As shown in FIG. 17, demographic information associated with the patient can be added. Such information can include a patient ID, which is associated with the patient's ID card, patient name, marital status, gender, race/ethnicity, date of birth, phone numbers, social security number, e-mail address, address, and the like. Data entry fields 1702 can be provide to allow the demographic information to be entered. Additional information that can be entered in the demographic section includes insurance information, emergency contact information, an ID card number, an ID card issue date, the name of the patient's primary physician, and a photograph of the patient using the data entry fields 1702.

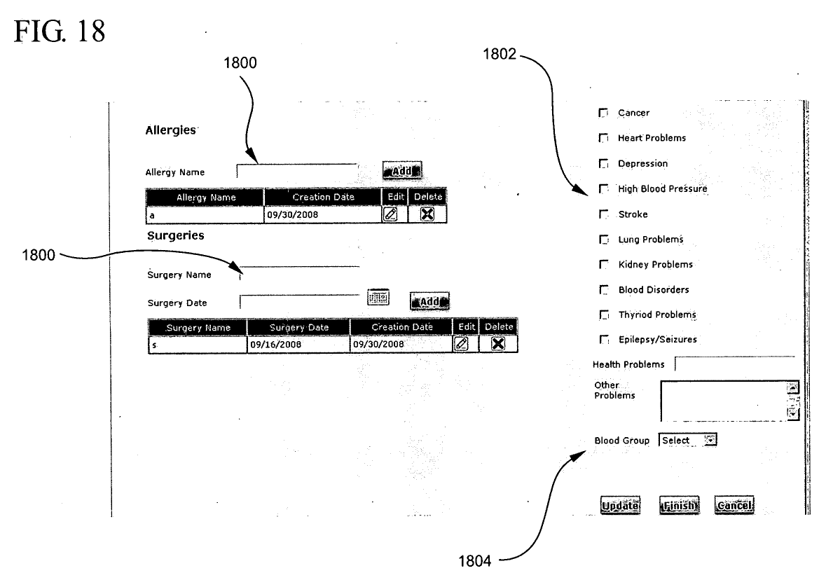

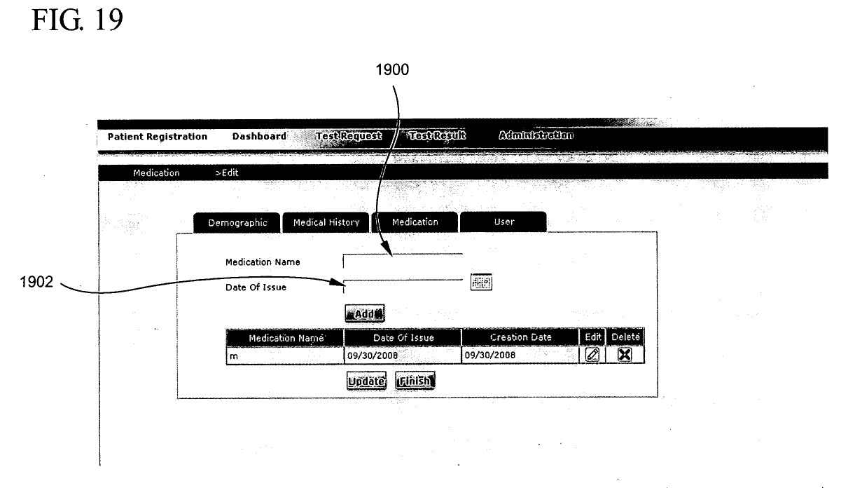

FIG. 18 is an exemplary screen shot that illustrates a screen for entering a medical history of the patient and FIG. 19 is an exemplary screen shot that illustrates a screen for entering patient medication information. The screen can include data entry fields 1800 for entering allergy information, and surgery information and can include check boxes 1802 for selecting ailments, diseases, or other health related issues associated with the patient. The patient's blood type is also preferably entered using a blood type selector 1804. As shown in FIG. 19, medication information associated with the patient can be entered using data entry fields 1900 and 1902.

FIGS. 20-22 are exemplary screen shots illustrating a test request/results portion of the web-based management application. Referring to FIG. 20, the test requests portion of the application allows, for example, a doctor or a clerk to enter tests to be conducted for a specified patient. A search can be performed based on patient information, such as the patient's name, the patient's ID, and the like, using the data entry fields 2000. The search can return a list 2002 of patients matching the searching criteria. In some embodiments, the search results can be limited to only display those patients that are associated with the doctor performing the search so that the doctor cannot access information about people who are not the doctor's patients.

Once the search results are returned, the doctor can schedule a date and time of the test using data entry fields 2004 and 2006, and can enter the name of the doctor requesting the test using a data entry field 2008. In addition, the doctor can specify in which of the medical diagnostics units the test are to be conducted using the cell name selector 2010. A list 2012 of diagnostic tests that can be performed can be displayed, and the doctor can select the test to be performed at the scheduled time. In some embodiments, the doctor must enter the patient's date of birth and gender to request tests.

After the patient has visited the medical diagnostics unit and the tests were performed, the doctor can review the test results by accessing a test results portion of the web-based management application. As shown in FIG. 21, to retrieve the test results, the doctor may again have to search for the patient using information associated with the patient. The doctor can narrow the search results to a particular time period using data entry fields 2100 and 2102. The search results can be displayed as a list 2104 to the doctor, who can select the appropriate patient and select a view button 2106 to begin reviewing the test results for test performed on the patient in the medical diagnostics unit, as shown in FIG. 22.

The results 2200 can be formatted in a list, table, graph, or other form suitable for conveying the test results 2200 to the doctor. The test results 2200 can identify measured health parameters of the patient and can compare the test results to expected or ‘normal’ results. Using the test results, the doctor can identify additional test to be performed or particular health-related parameters to be monitored by subsequent visits to the medical diagnostics unit or to the doctor's actual offices.

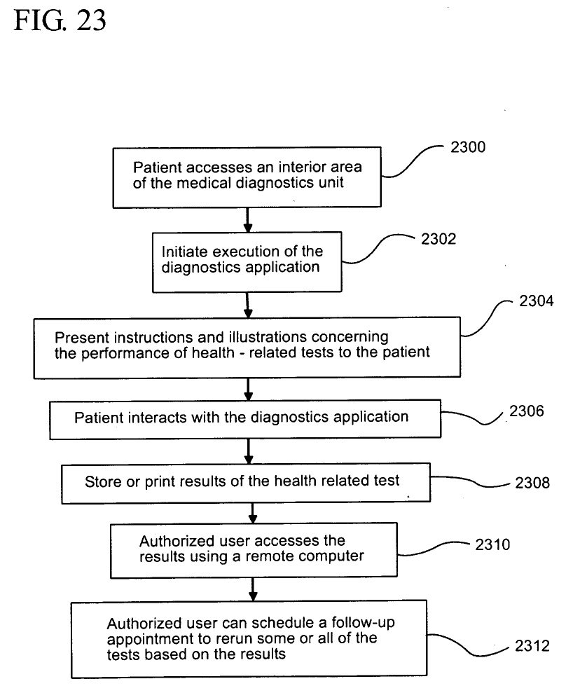

FIG. 23 is flowchart illustrating an exemplary implementation in accordance with embodiments of the present invention. A patient can access an interior area of the medical diagnostics unit via a locked door using a computer readable patient ID card, such as a smart card or bar coded card (step 2300). The patient can unlock the door by swiping or otherwise presenting the patient ID card to a card reader. The patient can receive the patient ID card from a doctor, nurse, administrator, vending unit, card printer, or other person or device. The patient ID card can hold information concerning the patient including, but not limited to a patient name, contact information, medical history, medications, allergies, insurance information, and the like. Once the patient has entered the medical diagnostics unit, the door can be closed and locked to prevent others from entering the medical diagnostics unit while the patient performs the test.

To begin the health-related testing process, the patient can again present the patient ID card to a card reader mounted in the interior of the medical diagnostics unit, which initiates the execution of the diagnostics application by the host computer (step 2302). Instructions and illustrations for each test can be presented to the patient using the VDU so that the patient understands how the test should be performed (step 2304). The user can interact with the diagnostics application using the VDU or other devices, such as a microphone, to select options presented by the diagnostics application (step 2304). As the patient performs the health-related tests, results from the tests can be stored in the host computer, a data storage system, and/or on the patient ID card, or can be printed without storing the results (step 2306).

For embodiments, where the tests are stored on the host computer or a data storage system, an authorized user can access the results using a remote computer (step 2308). Access to the results can be facilitated using the web-based medical management application. The access to the test results can be restricted based on a role of the authorized user to protect the patient's information. The web-based application can present the results to the authorized user and can allow the authorized user to schedule a follow-up appointment to rerun some or all of the tests (step 2310).

It is understood that the embodiments described herein can use hardware, software, or a combination of hardware and software. For example, embodiments can use a computer system configured to execute instructions of an application, which can control an operation of the computer system such that it carries out embodiments described herein. The computer system can be one or more computing devices, and in some embodiments the computer system can be implemented as a distributed system of networked computing devices. Alternatively, a specific use computer, containing specialized hardware for carrying out embodiments can be utilized.

Terms such as applications, computer program, software program, program, program product, software, etc., in the present context mean any expression, in any language, code or notation, of a set of instructions intended to cause a system having an information processing capability to perform a particular function either directly or after either or both of the following: (a) conversion to another language, code or notation; and/or (b) reproduction in a different material form.

While preferred embodiments of the present invention have been described herein, it is expressly noted that the present invention is not limited to these embodiments, but rather the intention is that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

What is claimed is:

1 A medical diagnostics apparatus comprising:
a portable enclosure including an access door dimensioned to permit a person to enter the enclosure, the enclosure forming an interior area enclosed by a platform and walls to promote patient privacy;
a plurality of medical test units for conducting health-related self-test measurements;
a video display unit comprising a touch-screen for providing instructions to a patient to operate the medical test units; and
a host computer adapted to receive and store results transmitted from the medical test units.

2. The medical diagnostics apparatus as defined in claim 1, wherein the plurality of medical test units are capable of determining height, weight, pulse, blood oxygen level, blood pressure, and lung capacity.

3. The medical diagnostics apparatus as defined in claim 1, wherein one of the medical test units is a blood pressure unit having a sphygmomanometer movably mounted on a track, the sphygmomanometer being configured to slide along the track fixed on a shelf in the enclosure.

4. The medical diagnostics apparatus as defined in claim 1, wherein one of the medical test units is an electrocardiogram (ECG) unit having transducers pivotally coupled to an underside of a chair in the enclosure via arms, the transducers configured to be positioned about a seat of the chair.

5. The medical diagnostics apparatus as defined in claim 4, wherein the arms are telescopically configured so that the transducers can be pushed away from the seat of the chair.

6. The medical diagnostics apparatus as defined in claim 1, wherein the ECG unit includes transducers operatively connected to a platform of the enclosure for receiving a patient's feet during an ECG test.

7. The medical diagnostics apparatus as defined in claim 1, wherein the medical test units include at least one of a urine analysis unit and a blood analysis unit.

8. The medical diagnostics apparatus as defined in claim 1, further comprising an integrated vending unit configured to vend a sealed container of items required for performance of health-related self test measurements associated with the medical diagnostics apparatus.

9. The medical diagnostics apparatus as defined in claim 1, wherein the enclosure is formed from body sections and ceiling sections, the body sections and the ceiling sections being dimensioned to fit through a doorway of an existing structure, the body sections and the ceiling sections capable of being assembled within the existing structure and having an assembled dimension that is larger than the doorway.

10. A medical diagnostics system comprising:
a medical diagnostics unit formed from a portable enclosure having an interior area defined by walls and an access door dimensioned to permit a person to enter the enclosure, the medical diagnostics unit including a plurality of medical test units for conducting health-related self-test measurements, a video display unit comprising a touch-screen for providing instructions to a patient to operate the medical test units, and a host computer adapted to receive and store results transmitted from the medical test units; and
a remote computer configured to access the results of the health-related self-test measurements using a web-based medical management application.

11. The medical diagnostics system as defined in claim 10, wherein the remote computer is configured to communicate with the host computer using the web-based medical management application to access the results.

12. The medical diagnostics system as defined in claim 10, further comprising a data storage system to receive and store the results from the host computer, wherein the remote computer is configured to communicate with the data storage system using the web-based medical management application to access the results.

13. The medical diagnostics system as defined in claim 10, further comprising a server configured to provide the web-based medical management application to the remote computer.

14. The medical diagnostics system as defined in claim 10, wherein access to the web-based medical management application and portions thereof are restricted to authorized users.

15. The medical diagnostics system as defined in claim 10, wherein access to portions of the web-based medical management application is restricted based on roles of the authorized users.

16. A method of conducting and recording health information of patient comprising the steps of:
providing a medical diagnostics unit which includes a plurality of medical test units for conducting health-related measurements mounted within an enclosure adapted to permit a patient to enter the unit;
testing the patient by conducting a plurality of health-related self-test measurements using the medical test units;
recording patient information obtained by use of the medical test units in a host computer located within the medical diagnostics unit; and
viewing the results of the testing by an authorized user at a location remote to the medical diagnostics unit.

17. The method as defined in claim 16, further comprising restricting viewing the results based on a role of the authorized user.

18. The method as defined in claim 16, further comprising restricting access to the medical diagnostics unit to patients having a computer readable personal identification (ID) card, the patient ID card being read by the medical diagnostics unit.
